(19) United States
(12) Patent Application Publication
(10) Pub. No.: US 2008/0208345 A1
(43) Pub. Date: Aug. 28, 2008

(54) Intervertebral implant

(75) Inventors: R. John Hurlbert, Calgary (CA); Stephen J. Duplessis, Calgary (CA); Lali Sekhon, Reno, NV (US)

Correspondence Address: Howard M. Ellis;SIMPSON & SIMPSON, PLLC, 5555 Main Street, Williamsville, NY, 14221, US

(73) Assignee: KINETIC SPINE TECHNOLOGIES, INC., Calgary (CA)

(21) Appl. No.: 12/150,137

(22) Filed: Apr. 25, 2008

Related U.S. Application Data
(63) Continuation of application No. PCT/CA2006/000176, filed on Oct. 27, 2006.
(60) Provisional application No. 60/730,901, filed on Oct. 27, 2005.

(51) Int. Cl. A61F 2/44

(52) U.S. Cl. 623/17.16; 623/17.15; 623/17.14

(57) ABSTRACT

An intervertebral implant, or disc prosthesis, comprises a pair of cooperating elements being provided in a generally “X” shaped structure. The elements are comprised of cooperating shells that are maintained separated by a resilient material provided therebetween. The elements allow for rotational and translational movement when implanted.

### CROSS REFERENCE TO PRIOR APPLICATIONS

The present application is a Continuation of PCT application no. PCT/CA2006/001769, filed Oct. 27, 2006, which claims priority from U.S. application No. 60/730,901, filed Oct. 27, 2005. The entire disclosures of these applications are incorporated herein by reference.

### FIELD OF THE INVENTION

The present invention relates to the field of spinal implants and, more particularly, to intervertebral implants, or disc prostheses, that are capable of percutaneous implantation.

### DESCRIPTION OF THE PRIOR ART

The spine is a complicated structure comprised of various anatomical components, which, while being extremely flexible, provides structure and stability for the body. The spine is made up of vertebrae, each having a ventral body of a generally cylindrical shape. Opposed surfaces of adjacent vertebral bodies are connected together and separated by intervertebral discs (or “discs”), comprised of a fibrocartilaginous material. The vertebral bodies are also connected to each other by a complex arrangement of ligaments acting together to limit excessive movement and to provide stability. A stable spine is important for preventing incapacitating pain, progressive deformity and neurological compromise.

The anatomy of the spine allows motion (translation and rotation in positive and negative directions) to take place without much resistance, but as the range of motion reaches physiological limits, the resistance to motion gradually increases to bring the motion to a gradual and controlled stop.

Intervertebral discs are highly functional and complex structures. They contain a hydrophilic protein substance that is able to attract water and thereby increase its volume. The protein material, also called the nucleus pulposis, is surrounded and contained by a ligamentous structure called the annulus fibrosis. The discs mainly perform load bearing and motion control functions. Through their weight bearing function, the discs transmit loads from one vertebral body to the next while providing a cushion between adjacent bodies. The discs allow movement to occur between adjacent vertebral bodies but within a limited range, thereby giving the spine structure and stiffness.

Due to a number of factors such as age, injury, disease etc., it is often found that intervertebral discs lose their dimensional stability and collapse, shrink, become displaced, or otherwise damaged, or degenerated. It is common for diseased or damaged discs to be replaced with prostheses and various versions of such prostheses, or implants, are known in the art. One of the known methods of treating damaged discs involves removal of the damaged disc and replacement with a spacer into the space occupied by the disc. However, such spacers also fuse the adjacent vertebrae together and, in the result, prevent any relational movement there-between. More recently, disc replacement implants that allow movement between adjacent vertebrae have been proposed. An example of such an implant is taught in U.S. Pat. No. 6,179,874.

Current surgical management of diseased discs involves open exposure of the disc space either through an anterior approach or a posterior approach, excision of all or most of the disc and either placement of a large single piece artificial disc or interbody fusion with bone graft, cages, or some similar substitute for the disc space. These latter procedures are invasive and are still plagued with deficiencies such as, inter alia, access problems, imaging issues, and difficulty in replacement or adjustment.

Thus, there exists a need for an intervertebral disc implant that overcomes at least some of the deficiencies in the prior art solutions. More particularly, there exists a need for a spinal implant that has the following features:

the ability to be placed, or implanted, through a small incision.

the ability to be easily replaced or adjusted.

the ability to be clearly observed on postoperative imaging.

the ability to be implanted as an outpatient procedure.

resistance to being dislodged or subluxed.

### SUMMARY OF THE INVENTION

In one aspect, the present invention provides an implant for replacing intervertebral discs.

In another aspect, the invention provides an artificial intervertebral implant, or disc, that is capable of subcutaneous implantation, replacement or adjustment.

Thus, in one aspect, the invention provides an intervertebral disc prosthesis comprising:

first and second cooperating elements, at least a portion of the first element overlapping a portion of the second element to provide inter-engagement therebetween;

the first and second elements being moveable with respect to each other in rotational and translational directions;

the first and second elements each comprising generally elongate bodies whereby, when the first and second elements are engaged, the disc comprises a generally “X” shaped structure.

### BRIEF DESCRIPTION OF THE DRAWINGS

The features of the invention will become more apparent in the following detailed description in which reference is made to the appended drawings wherein:

FIG. 1 is a schematic illustration of the range of motion of a spinal vertebra.

FIG. 2a is side elevation of an inner wing according to an embodiment of the invention.

FIG. 2b is side elevation of an outer wing according to an embodiment of the invention.

FIG. 3a is side elevation of an inner wing according to another embodiment of the invention.

FIG. 3b is side elevation of an outer wing according to another embodiment of the invention.

FIG. 4 is an end elevation of an outer wing illustrating the stabilizing keels of the invention.

FIG. 5a is a side elevation of another embodiment of the inner wing of FIG. 2a.

FIG. 5b is a side elevation of another embodiment of the outer wing of FIG. 2b.

FIG. 6a is a side elevation of another embodiment of the inner wing of FIG. 3a.

FIG. 6b is a side elevation of another embodiment of the outer wing of FIG. 3b.

FIGS. 7a to 7c are side elevations of the wings of FIGS. 2a and 2b in various orientations.

FIGS. 8a to 8c are side elevations of the wings of FIGS. 3a and 3b in various orientations.

FIG. 9 is a plan view illustrating the placement of the present invention.

FIG. 10 is a plan view radiograph of a vertebrae illustrating the placement of the present invention.

### DETAILED DESCRIPTION OF THE INVENTION

In the following description, the terms “superior”, “inferior”, “anterior”, “posterior” and “lateral” will be used. These terms are meant to describe the orientation of the implants of the invention when positioned in the spine. Thus, “superior” refers to a top portion and “posterior” refers to that portion of the implant (or other spinal components) facing the rear of the body when the spine is in the upright position. It will be appreciated that these positional terms are not intended to limit the invention to any particular orientation but are used to facilitate description of the implant.

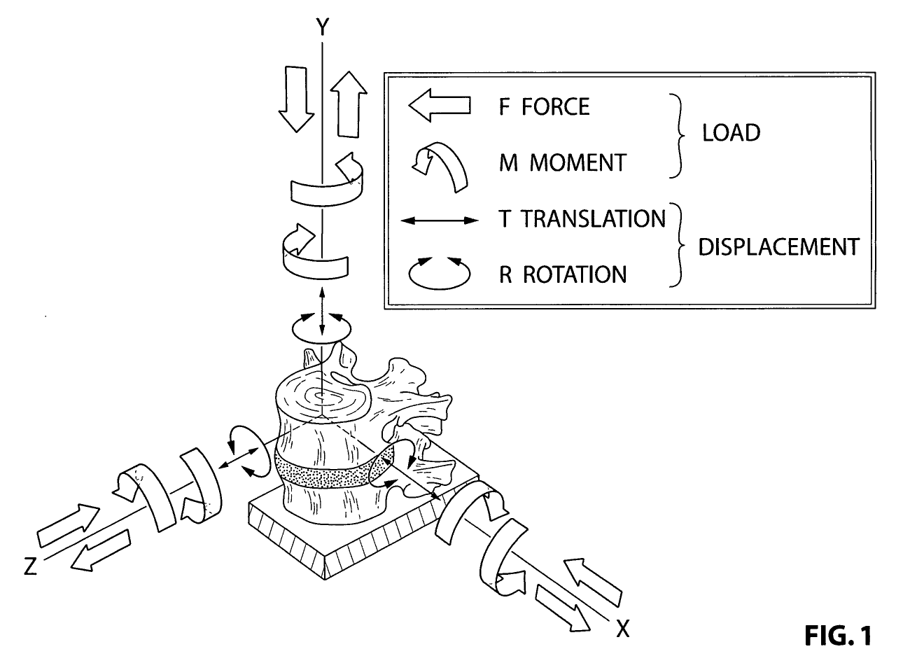

FIG. 1 illustrates the complexity of vertebral movement by indicating the various degrees of freedom associated therewith. In the normal range of physiological motion, vertebrae extend between a “neutral zone” and an “elastic zone”. The neutral zone is a zone within the total range of motion where the ligaments are relatively non-stressed; that is, the ligaments offer relatively little resistance to movement. The elastic zone is encountered when the movement occurs at or near the limit of the range of motion. At this zone, the visco-elastic nature of the ligaments starts providing resistance to the motion thereby limiting same. The majority of everyday motion occurs within the neutral zone and only occasionally continues into the elastic zone. Motion that is contained within the neutral zone does not stress soft tissue structures whereas motion into the elastic zone will cause various degrees of elastic responses. Therefore, in the field of spinal implants in particular, by restricting motion to the neutral zone, stresses to adjacent osseous and soft tissue structures will be minimised. For example, such limitation of movement will reduce facet joint degeneration.

The present invention provides artificial discs or implants for replacing intervertebral discs that are damaged or otherwise dysfunctional. In general terms, the present invention provides a spinal implant for replacing intervertebral discs and that are primarily designed to be subcutaneously implantable. The implant of the invention is generally comprised of interlocking sections that are moveable relative to each other and that contain resilient, force-absorbing nuclei.

Basic Structure of Implant

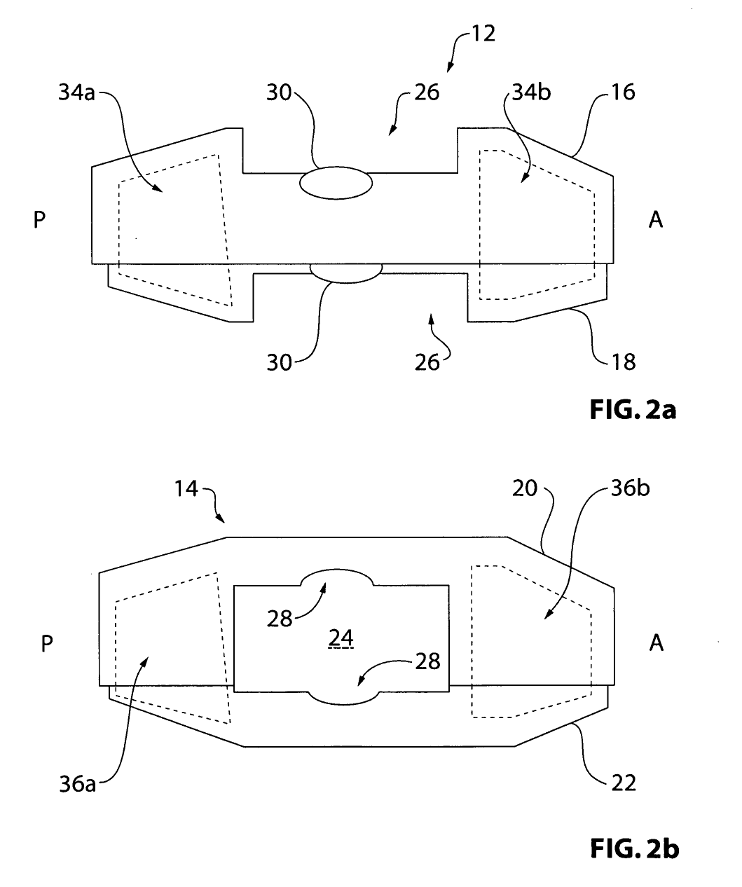

In one aspect, the implant of the invention consists of two interlocking sections with one section (referred to as the “inner wing”) extending through the other (referred to as the “outer wing”). FIGS. 2a and 2b illustrate the basic structure of each of the inner 12 and outer 14 wings, respectively. Each of the wings have anterior and posterior ends indicated at “A” and “P”, respectively. As shown, each of the inner and outer wings, 12 and 14, are comprised of cooperating superior and inferior shells. Thus, superior and inferior shells 16 and 18 combine to form inner wing 12 while superior and inferior shells 20 and 22 combine to form outer wing 14. As illustrated, the superior shells 16 and 20 are preferably designed to overlap the respective inferior shells 18 and 22 to allow for an extended range of motion with some constraint (e.g. rotation). In one aspect, the superior shells may overlap the inferior shells by several millimetres although the extent of such overlap will depend on several factors as will be discussed below. The respective pairs superior and inferior shells do not need to be connected to each other since, once implanted, the load placed on the pairs will be sufficient to maintain their association. However, in order to assist in maintaining the paired structure prior to implantation, the pairs of shells may be connected by means of hooks, ridges and the like (as will be apparent to persons skilled in the art) to prevent separation of the shells while permitting compression there-between.

As indicated above, the inner wing 12 is designed to fit into the outer wing 14. For this purpose, the outer wing 14 is provided with an aperture 24 into which the inner wing 14 can be inserted. The inner wing 14 is in turn provided with recesses 26a and 26b in the superior and inferior shells 16 and 18, respectively, to facilitate the positioning of the inner wing 14 within the aperture 24. Thus, the recess 26a is provided in the superior shell 16 of the inner wing 14 and engages the portion of the aperture 24 formed by the superior shell 20 of the outer wing. Similarly, recess 26b, provided in the inferior shell 18 of the inner wing 14 engages the portion of the aperture 24 formed by the inferior shell 22 of the outer wing. In a further preferred embodiment, the superior and inferior walls of aperture 24 are provided with at least one recess 28 to receive a cooperatively shaped projection 30 provided on the superior and inferior surfaces of the recesses 26a and 26b. As will be appreciated, the recesses 28 and projection 30 serve to location and position the outer and inner wings when engaged. In this regard, the projections 30 and recesses 28 are designed and sized to provide a relatively tight interference fit when the wings are assembled to form the assembled implant. Such a “ball and socket” arrangement between the projections 30 and recesses 28 also serve as pivot points for relative rotation and tilting movements between the inner and outer wings.

As described further below, when the implant of the invention is to be positioned within the spine, the outer wing 14, consisting of its two shells 20 and 22, would be initially implanted followed by the inner wing 12. The latter would be placed on its side and passed through the aperture 24 before being turned 90° to sit in the upright position. In such position, the inner wing 12 will be interlocked with the outer wing 14. As will be understood, such interlocking will be assisted by engaging the projections 30 into the respective recesses 26a and/or 26b.

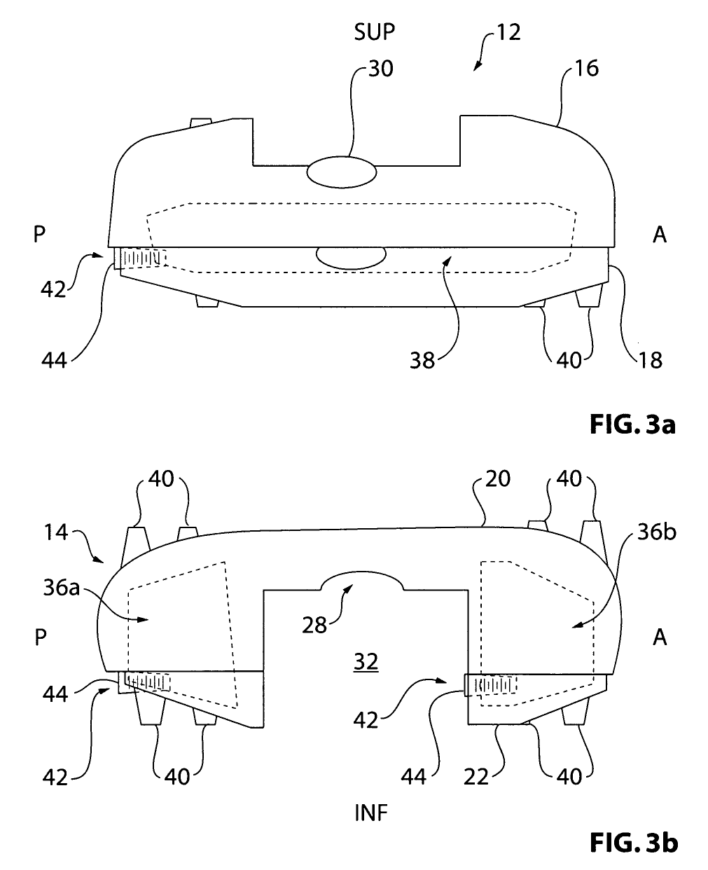

FIGS. 3a and 3b illustrate another embodiment of the inner and outer wings described above where like elements are referred to with like reference numerals. In this case, the aperture 24 of the outer wing 14 is replaced by a gap 32 that extends through the inferior shell 22 of the outer wing 14. In turn, the inner wing 12 is provided with only one recess 26 to engage the gap 32. Thus, during implantation of the embodiment shown in FIGS. 3a and 3b, the inner wing 12, would be pushed under the outer wing 14 with no rotation required.

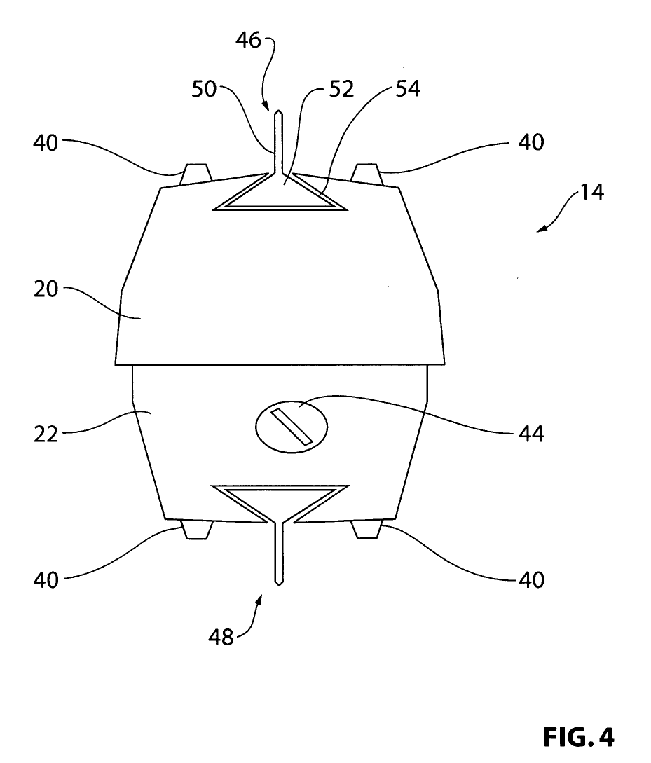

As shown in FIGS. 2a,b and 3a,b, the external surface of the superior shells 16 and 30 may be either angled (as shown in FIGS. 2a,b) or smooth (as shown in FIGS. 3a,b). FIG. 4 illustrates an outer wing 14 of FIG. 2b in an end view. This Figure also illustrates the overlap of the superior shell 20 over the inferior shell 22. FIG. 4 also shows other embodiments of the invention as discussed further below.

Inner Cavities

As shown in FIGS. 2a and 2b, the respective pairs of superior and inferior shells, 16 and 18, 20 and 22, are provided with cooperating cavities such that, when the shells are combined, generally closed reservoirs 34a, 34b, 36a, and 36b are formed in the wings 12 and 14. As shown, reservoirs 34a and 36a are provided in the posterior ends of the wings while reservoirs 34b and 36b are provided in the anterior ends.

Within each of the reservoirs 34a,b and 36a,b, is provided a nucleus (not shown) formed from a resilient material such as a hydrogel or other similar material as will be known to persons skilled in the art. The nucleus serves to separate the respective superior and inferior shells from each other and to absorb any compressive forces applied against same. In the embodiment shown in FIGS. 3a and 3b, the reservoir 38 for the nucleus in the inner wing 12 would generally extend over the length of the inferior shell 18.

In the embodiments illustrated in FIGS. 2a,b and 3a,b, the reservoirs 34a,b and 36a,b are provided with a generally trapezoidal shape, when viewed in cross section. It is believed that such a design is preferred in order to maximise the available volume of the respective wings and, therefore, allow for nuclei of larger volume. It will be understood that a larger nucleus will provide increased energy absorption. The generally trapezoidal shape is the result of the required tapering of the ends of each wing. It will be understood, however, that the aforementioned reservoirs and nuclei may be provided in any shape while still providing the needed energy absorbing capability.

Access Portals To Hydrogel Reservoirs

FIGS. 3a and 3b also show another embodiment of the invention wherein access ports 42 are provided for allowing access to the reservoirs 36a and 36b that contain the nuclei. These access ports 42 may be maintained closed by, for example, a screw 44. It will be understood that, in such case, the ports 42 will be provided with an appropriately threaded wall to engage such screws. The screws 44 are shown in side view in FIGS. 2a,b and in end view in FIG. 4. Such screws 44 serve to allow for access to the reservoirs containing the above mentioned nuclei in the event that such access is needed post-implantation. For example, such access may be required when one or more of the nuclei need to be removed and/or replaced. As shown in FIG. 3b and as will be understood by persons skilled in the art, the ports 42 are designed to face the posterior end of the implant so as to allow for in-situ access to the nuclei reservoirs after implantation. In this regard, it will also be understood that the port 42 located at the anterior (A) end of the inferior shell 22 of the outer wing 14 would be angled off the midline with respect to the longitudinal axis of the implant so as to allow for easier access thereto when the implant is in position in the spine.

Stabilising Studs and Outer Coatings

In another aspect of the invention, as illustrated in FIGS. 3a, 3b and 4, the outer surfaces of the inner and outer wings, 12 and 14, may be provided with stabilizing studs 40 to facilitate initial stability of the implant when initially positioned within the spine. Preferably, two to six studs 40 will be provided on the leading and trailing edges (i.e. the anterior and posterior ends) of the inner and outer wings. More preferably, as shown in FIG. 3a, the leading edge (i.e. anterior end) of the superior shell 16 of the inner wing 12 would have no studs in order to prevent any hindrance during insertion of the inner wing 12 through the gap 32 of the outer wing 14. The studs 40 provide one type of initial stability for the implant of the invention by preventing migration of the implant after insertion and promoting incorporation of the superior and inferior shells into surrounding endplate of adjacent vertebrae. As illustrated in FIG. 4, it will be appreciated that studs 40 can also be provided on the embodiment of the wings of FIGS. 2a and 2b.

In another aspect, the outer surfaces of the shells of the inner and outer wings may be coated with a porous material to allow for bony ingrowth. In addition, such surfaces may be provided with bone morphogenic proteins as well to encourage assimilation of the implant into the neighbouring spinal structures.

Stabilizing Keels

As indicated above, the outer surfaces of the superior shells 16 and 20 of the inner and outer wings (12, 14), respectively, can be provided with stabilizing studs 40 for assisting in maintaining the implant in position soon after implantation. FIG. 4 illustrates another embodiment of the invention wherein such stabilization can be achieved with stabilizing keels 46 and 48, provided, respectively, on the superior shell 20 and inferior shell 22 of the outer wing 14. As shown in FIG. 4, keel 46 includes a generally vertically extending flange 50 and a base 52 having a flared section opposite the flange 50. The base 52 is embedded within a track 54 provided on the upper surface of the superior shell 20 such that the keel 46 is inseparable from the superior shell 20. As illustrated in FIG. 4, the track 54 is preferably larger in size than the base 52 whereby the keel 46 is able to move laterally within a limited range, such range being bounded by the opening of the track 54. As shown, the keel 48 provided on the inferior shell 22 will have generally the same structure and arrangement as that for keel 46.

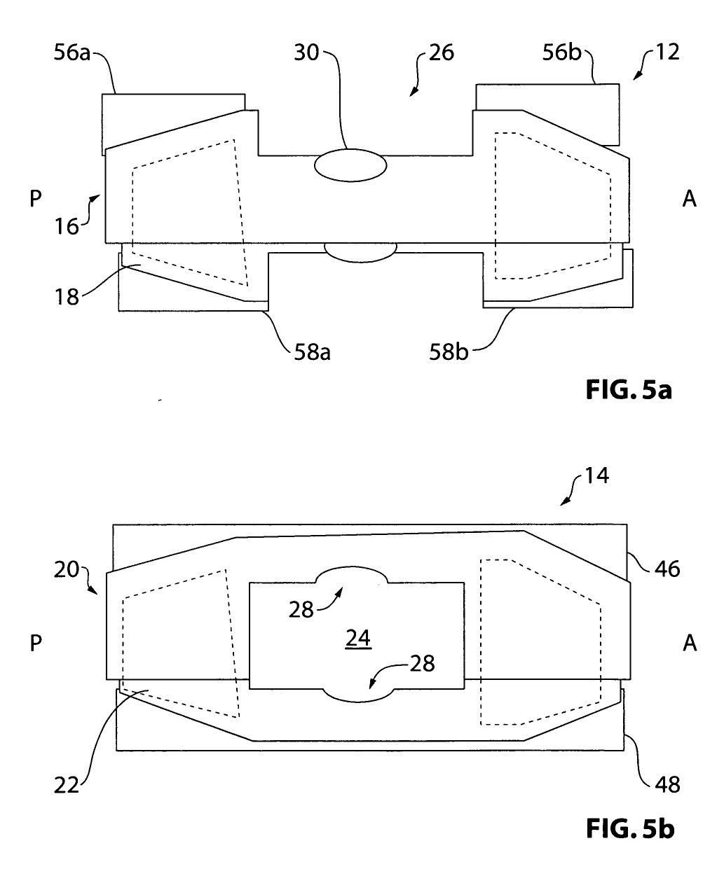

FIG. 5b illustrates the outer 14 wing of FIG. 4 in a side elevation. As mentioned above, the outer wing 14 shown in FIGS. 4 and 5b is similar to the outer wing 14 depicted in FIG. 2b but with the superior 20 and inferior 22 shells being provided with the aforementioned keels 46 and 48, respectively. In a similar manner, FIG. 5a illustrates the inner wing 12 of FIG. 2a wherein stabilizing keels 56 and 58 are provided. Due to the presence of the gap 26 on both the superior 16 and inferior 18 shells of inner wing 12, the respective keels are divided into section 56a,b and 58a,b. However, the structure and function of the latter keels is substantially the same as keel 46 described in detail above.

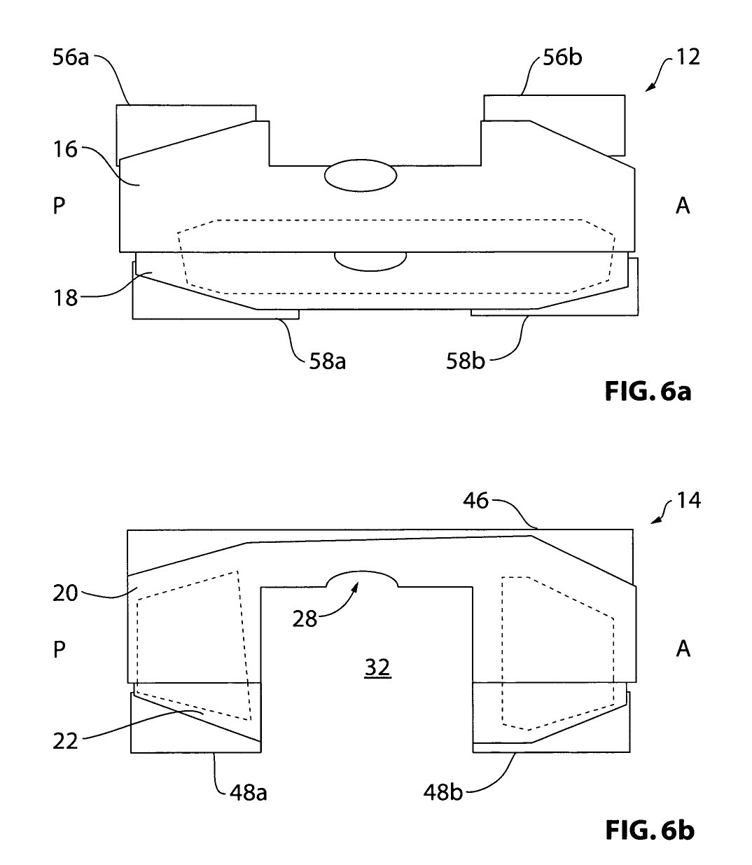

FIGS. 6a and 6b illustrate, generally, the configuration of the inner and outer wings of FIGS. 3a and 3b but with some differences. For example, it is noted that although the interaction mechanism between the inner 12 and outer wings 14 is the same (that is the outer wing 14 is provided with a gap 32 to accommodate the inner wing 12), it is noted that the outer surface of the shells is angular as in FIGS. 2a and 2b. Further the wings 12 and 14 of FIGS. 6a,b are noted as including stabilizing keels. In this case, the stabilizing keels of the inner wing 12 are similar to those of FIG. 5a. However, since the upper wing 14 of FIG. 6a includes a gap 32, the keel provided thereon is divided into two section 48a and 48b.

The keels described above would preferably be cut through the endplate of the adjacent vertebrae and could be added after placement of the inner and outer wings. It will be understood that by providing the stabilizing keels of the inner wing in two sections, as shown in FIGS. 5a and 6a, the articulating mechanism between the inner and outer wing would not be compromised.

As will be appreciated, when the implant includes the keels referred to above, the inner wing should first be inserted through the outer wing prior to installing the keels on the inner wing. Thus, in one embodiment, the implant of the invention may be positioned in the following manner. First, the outer wing is positioned in the desired location followed by insertion of the inner wing there-through and rotation of the inner wing into the desired position. Following this, the anterior facing keels (superior and inferior) of the inner wing are added followed by placement of the superior and inferior full length keels of the outer wing. Finally, the posterior keels of the inner wing are added. It will be appreciated that the above description is one method of implantation and that various others will be apparent to persons skilled in the art.

The aforementioned keels may be made of a variety of materials as will be apparent to persons skilled in the art. Generally, the keels should be made of a rigid material or a flexible material having some degree of rigidity to provide the required stability. In a preferred embodiment, the keels are made from titanium or PEEK (i.e. polyether-etherketone or polyaryletherketone).

Angulation

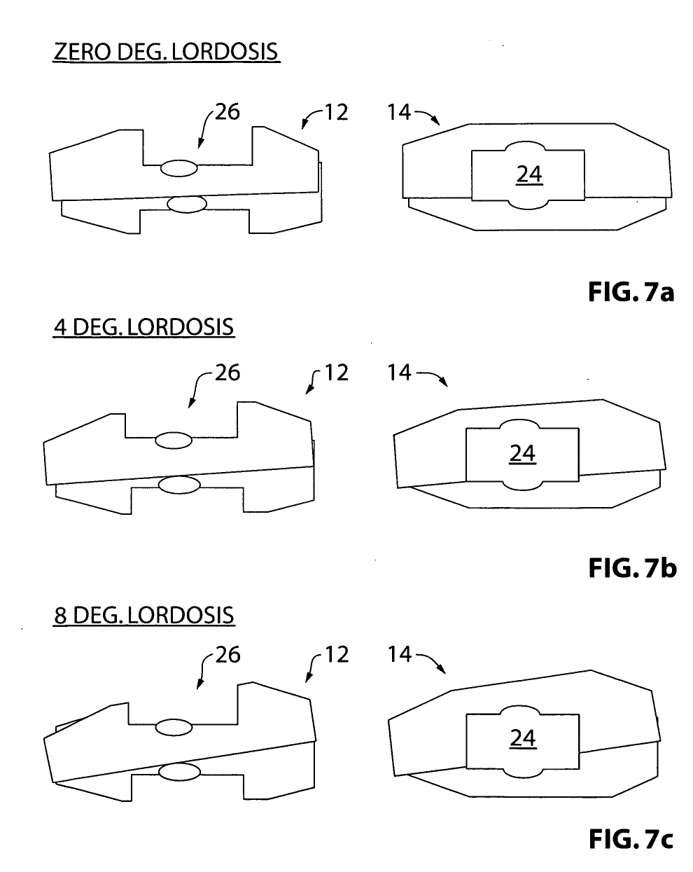

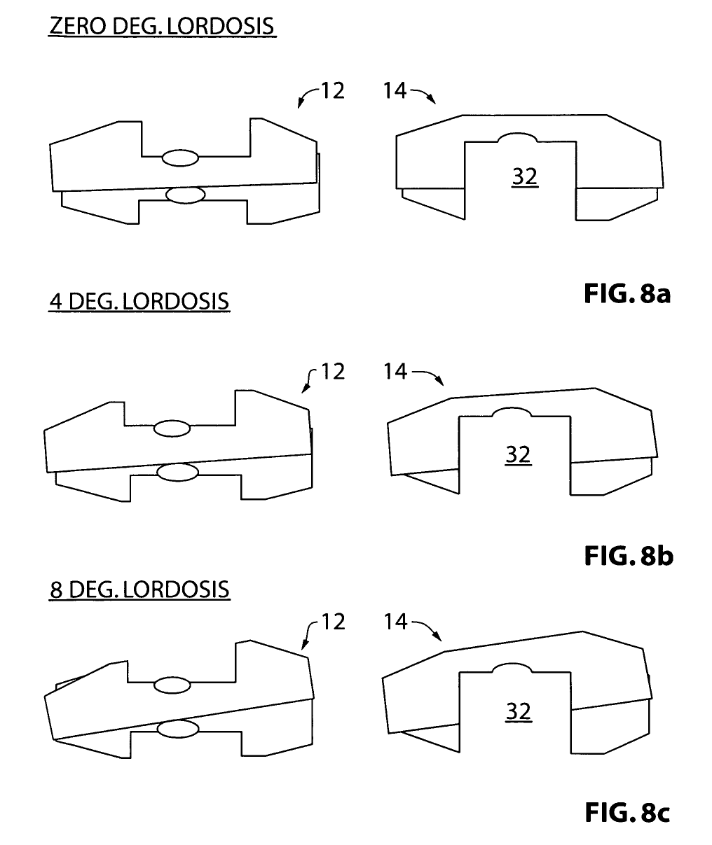

The implants of the present invention can be formed to provide any desired angular positioning of the wings so as to allow for variable disc space angulations. In this way, the implants of the invention can accommodate, for instance, the maintenance or restoration of lordosis (i.e. natural curvature of the spine). FIGS. 7a to 7c illustrate a few sample angular orientations of the wings 12 and 14 of FIGS. 2a and 2b, wherein the angle of articulation between the superior and inferior shells is varied between 0°, 4° and 8°. Similarly, FIGS. 8a to 8c illustrate the same angular orientations of the wings 12 and 14 of FIGS. 3a and 3b

Anatomical Placement

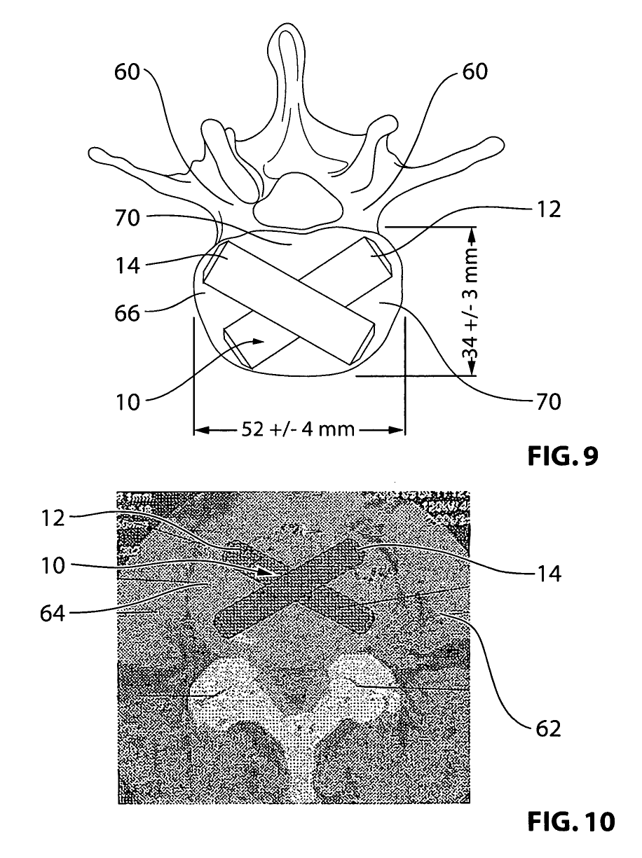

FIGS. 9 and 10 illustrate the placement of the implant within the spine as well the interlocking of the two wings. As shown, in its implanted form, the implant of the invention assumes a generally “X” shaped arrangement when viewed in plan. The arms of the “X” shape are formed by the wings 12 and 14. As indicated above, the implant of the present invention is designed for percutaneous implantation thereby involving a minimally invasive procedure. Prior to implantation, the disc space would be entered percutaneously and the disc space cleaned along the trajectory of the implant so as to facilitate the insertion thereof. Following this, the endplates of adjacent vertebrae are decorticated. This phase of the procedure may be performed with, for example, image-guidance apparatus. However, it will be understood that any known methods may also be used. Once a channel is cleared for the insertion of the implant, each of the inner and outer wings of the device would be inserted and the inner wing interlocked with the outer wing. As illustrated in FIGS. 9 and 10, the implant 10 of the invention is implanted in a corridor lateral to the pedicle 60 and medial to the psoas muscle 62. The exiting root would be retracted superiorly. The implant 10 would be positioned in the disc space 64 on the apophyseal ring 66 extending from cortical endplate posteriorly to endplate anteriorly. In other words, the implant overlaps disc space from the posterior cortical rim to the anterior cortical rim. In this manner, the implant will be anchored on either side by resting on the denser apophyseal ring thereby avoiding subsidence which may be encountered if the implant was solely resting on cancellous bone 70.

FIG. 10 illustrates the paucity of the prosthesis of the invention adjacent to the neural structures. Such arrangement reduces the amount of artifact on imaging. As will be understood by persons skilled in the art, the percutaneous implantation made possible by the present invention, and by avoiding a true anterior retroperitoneal or transperitoneal approach, allows the preservation of the anterior annulus and generally retains the normal physical characteristics of this corridor. This therefore allows for possible future approaches through non-scarred tissue.

It will be understood that the inner and outer wings would come in different heights, lengths and widths to allow for restoration of disc space height and maximal endplate coverage. In this way, the present invention can be sized to fit within a range of disc space sizes.

Functional Mechanism of the Invention

Once the disc (i.e. prosthesis) of the invention is implanted, articulation can occur between the respective superior and inferior shell on each side of the implant, with the nuclei allowing for motion on each arm. This therefore, allows for flexion, extension, lateral flexion to each side, cushioning and rotation through either coupling of above motions or via sliding of the superior on the inferior shells. The bony ingrowth discussed above would anchor the respective inferior and superior shells to the respective endplate of the adjacent vertebrae.

Extension of Indications

As a percutaneously placed interlocking device, the disc of the present invention could also be used as a standalone interbody cage. In this embodiment, both the outer and inner wings would be hollow to allow for containment of bone graft of its equivalent with open apertures on all sides to allow for bony ingrowth. In addition, the superior parts of the implant and the medial and lateral walls would preferably be porous to allow for bony ingrowth. The initial stability would be provided by the stabilizing studs and wings but potentially this could be used as a standalone device. In this case, the above mentioned articulation would not be present.

The disc of the present invention could be provided in either two pieces or one piece. The disc of the invention can be made with a variety of materials as will be known to persons skilled in the art. For example, the endplates and annulus sections may be manufactured from steel, stainless steel, titanium, titanium alloy, porcelain, plastic polymers, PEEK or other biocompatible materials. The nuclei may comprise mechanical springs (for example made of metal), hydraulic pistons, a hydrogel or silicone sac, rubber, or a polymer or elastomer material.

### SUMMARY OF FEATURES OF THE INVENTION

As described above, the present invention comprises a unique percutaneously implantable intervertebral disc replacement that allows for a unique four-armed articulation that mimics normal intervertebral disc motion. By varying the location and the height of the resilient nuclei, the axis of rotation of the disc (i.e. prosthesis) can be varied as desired.

The various interlocking mechanisms of the two sections (i.e. wings) of the invention allow for a coupling of motion and load sharing as well resisting migration or expulsion of the device after implantation.

As discussed above, the disc of the present invention includes a unique “staged” implantation system, including initial implantation of the inner and outer wings, chiseling of the pathway for the stabilizing keels to be inserted, and placement of the stabilizing keels in one or more pieces, as needed, as the final step. In addition, the shape of the inner and outer shells with studs located on the anterior and posterior portions or superior and inferior wings, with the exception of the leading wing of the inferior shell, would facilitate the locking of the two devices as well as allowing for initial stability by anchoring the devices into the adjacent endplates.

In one embodiment, the inner and outer wings would be mismatched in size with an overlap of the outer wing on the inner wing. Such an overlap would allow, inter alia, for some degree of movement between the respective superior and inferior shells with a degree of rotation possible between the superior and inferior wings. The shells would act as a hard stop to further motion.

The screw threaded apertures allowing access to the nuclei receptacles would allow for unique in situ extraction and/or replacement of the nuclei through a percutaneous approach. The floating nucleus complex would allow for coupling of flexion/extension and axial rotation with lateral bending mimicking physiological movement.

Coupling of lateral angulation and lateral (coronal) translation with lateral bending would occur until the hard stop of the superior shell hitting the inferior shell was encountered.

The generally trapezoidal shape of one embodiment of the resilient nucleus (when viewed in cross section) is believed to allow maximum durability under loads of eccentric compression from directions other than true axial loading. In general, the nucleus cavities or receptacles are designed to be larger than the nuclei themselves. It will be understood the resulting such extra space in the receptacles allows for lateral expansion during compression of the nucleus such as during axial loading of the disc. The nuclei are preferably formed from a hydrogel but may be combination of mechanical springs or other compressible substance as well. It will be appreciated that the nuclei will preferably have load and displacement characteristics that are approximate those of a normal disc.

The device isolates axial rotation, lateral bending, flexion/extension into component vectors. The device reproduces neutral zone and elastic zone properties of an intact disc for individual vectors for each degree of freedom. The device allows for unconstrained and partially constrained coupled movements making use of engineered end-points (superior on inferior shell) that prevent excessive or non-physiological movement. Fully constrained stop mechanisms ensure the elastic zone is not exceeded, thereby preventing disc failure.

The footprint of disc is preferably maximized in both coronal and sagittal planes to help eliminate subsidence. The discs of the present invention can be provided in many sizes and heights to accommodate various sizes of discs in the normal spine. The placement of the implant on the outer apophyseal ring ensures reduced incidence of subsidence.

With the present invention, total disc removal would not be required. The chief action of the implant of the invention would be restoration of disc height and preservation of normal motion.

Although the invention has been described with reference to certain specific embodiments, various modifications thereof will be apparent to those skilled in the art without departing from the purpose and scope of the invention as outlined in the claims appended hereto. The disclosures of all prior art recited herein are incorporated herein by reference in their entirety.

1-10. (canceled)

11: An intervertebral disc prosthesis comprising:
a first cooperating element having a generally elongate body; and,
a second cooperating element having a generally elongate body, wherein at least a portion of the first cooperating element overlaps a portion of the second cooperating element to provide inter-engagement therebetween, the first and second cooperating elements being moveable with respect to each other in rotational and translational directions, and the first and second cooperating elements are arranged such that the disc comprises a generally “X” shaped structure when the first and second cooperating elements are engaged.

12: The intervertebral disc prosthesis as recited in claim 1 wherein the first cooperating element includes an aperture through which the second cooperating element extends.

13: The intervertebral disc prosthesis as recited in claim 2 wherein the second cooperating element includes at least one aperture to engage a portion of the aperture of the first cooperating element.

14: The intervertebral disc prosthesis as recited in claim 1 wherein the first and second cooperating elements include cooperating recesses, and wherein the recess of the first cooperating element is received within the recess of the second cooperating element.

15: The intervertebral disc prosthesis as recited in claim 1, wherein the first cooperating element comprises a first shell, having at least one cavity, and a second shell, having at least one cavity, wherein the second cooperating element comprises a first shell, having at least one cavity, and a second shell, having at least one cavity, wherein the shells are inter-engageable and arranged such that when the first and second shells are combined, the respective cavities combine to form a reservoir in the respective cooperating element, the reservoir being provided with a generally resilient member.

16: The intervertebral disc prosthesis as recited in claim 5 wherein each of the first and second cooperating elements includes at least one of the reservoirs.

17: The intervertebral disc prosthesis as recited in claim 6 wherein each of the first and second cooperating elements includes a pair of reservoirs, wherein one reservoir is provided on each end of the cooperating elements.

18: The intervertebral disc prosthesis as recited in claim 7 wherein the first shell is arranged to overlap the second shell.

19: The intervertebral disc prosthesis as recited in claim 8 wherein the first and second cooperating elements each includes an outer surface having one or more anchoring mechanisms adapted to anchor the disc to adjacent bony surfaces when implanted.

20: The intervertebral disc prosthesis as recited in claim 9 wherein the anchoring mechanisms are selected from the group consisting of stabilizing studs, stabilizing keels, a porous surface, or a combination thereof.
